(19) United States
(12) Patent Application Publication
(10) Pub. No.: US 2024/0156608 A1
(43) Pub. Date: May 16, 2024

(54) IMPLANT AUGMENTATION SYSTEMS AND METHODS OF USE

(71) Applicant: Paragon 28, Inc., Englewood, CO (US)

(72) Inventors: Luciano Bernardino BERTOLOTTI, Denver, CO (US); Gregory J. KOWALCZYK, Little Silver, NJ (US); James Keith DEORIO, Durham, NC (US)

(73) Assignee: Paragon 28, Inc., Englewood, CO (US)

(21) Appl. No.: 18/416,258

(22) Filed: Jan. 18, 2024

Related U.S. Application Data
(63) Continuation of application No. PCT/US2022/073829, filed on Jul. 18, 2022.
(60) Provisional application No. 63/223,640, filed on Jul. 20, 2021.
(60) Provisional application No. 63/263,615, filed on Nov. 5, 2021.

(51) Int. Cl. A61F 2/38; A61F 2/30; A61F 2/46

(52) U.S. Cl. CPC A61F 2/389; A61F 2/30734; A61F 2/4603; A61F 2002/30011; A61F 2002/30212; A61F 2002/30326; A61F 2002/30329; A61F 2002/30604; A61F 2002/30736

(57) ABSTRACT

The present disclosure relates to a system for augmentation of a tibial component. The system includes an augmentation component having a first porous structure, a second porous structure, a top portion having a top surface, and a bottom portion having a bottom surface arranged opposite the augmentation component from the top surface. The system also includes a tibial component and an instrument, with the tibial component being configured to couple to the augmentation component. The instrument includes an engagement portion having a geometry complimentary to that of the augmentation component. Further, a method of augmenting a tibial component is disclosed with the steps of collecting imaging data from a patient, identifying a void in the distal tibia, obtaining an augmentation component that corresponds to the void, coupling the augmentation component to the tibial component and implanting the tibial component into the tibia so the augmentation component occupies the tibial void.

### CROSS REFERENCE TO RELATED APPLICATIONS

This application is a continuation of International Application No. PCT/US2022/073829 filed Jul. 18, 2022, and entitled Implant Augmentation Systems and Methods of Use, which claims priority benefit under 35 U.S.C. § 119(e) to U.S. Provisional Application No. 63/223,640 filed Jul. 20, 2021; entitled Prosthetic Extension Stability Device and Hand Reamer, and U.S. Provisional Application No. 63/263,615 filed Nov. 5, 2021; entitled Stem Augmentation Device Implant and Method of Use. All of these applications are hereby incorporated herein by reference in their entireties.

### FIELD OF THE INVENTION

The present disclosure relates to surgical instruments, guides, and methods of use to be implemented in surgical procedures. The present disclosure relates to podiatric and orthopedic surgical instruments, guides, and methodology to be implemented in various procedures of the foot and/or ankle, for example arthroplasty. More specifically, but not exclusively, the present disclosure relates to surgical instruments, guides to be implemented in conjunction with instruments (as well as other components, for example implants, devices, systems, assemblies, etc.) and methods of use for performing ankle arthroplasty procedures.

### BACKGROUND OF THE INVENTION

Many currently available surgical instruments and implants, as well as methodology, do not completely address the needs of patients. Additionally, many currently available surgical instruments, implants, and methodology fail to account for properties of the ankle joint of various patients and accordingly can decrease favorability of the outcome for said patients.

### SUMMARY

The present disclosure is directed toward surgical instruments, implants, and methods directed to arthroplasty procedures.

A first aspect of the present disclosure is a system for augmentation of a tibial component. The system includes an augmentation component having a first porous structure, a second porous structure, a top portion having a top surface, and a bottom portion having a bottom surface arranged opposite the augmentation component from the top surface. The system also includes a tibial component and an instrument. The instrument includes an engagement portion having a geometry complimentary to that of the augmentation component.

According to the first aspect of the present disclosure, the augmentation component is couplable with a surface of the tibial component.

According to the first aspect of the present disclosure, the first porous structure is positioned proud relative to the second porous structure.

According to the first aspect of the present disclosure, the first porous structure includes a first porosity and the second porous structure includes a second porosity.

According to the first aspect of the present disclosure, the first porosity is greater than the second porosity.

According to the first aspect of the present disclosure, the top portion of the augmentation component is positioned superior relative to the bottom portion of the augmentation component.

According to the first aspect of the present disclosure, the bottom portion includes at least one first lateral dimension and the top portion includes at least one second lateral dimension.

According to the first aspect of the present disclosure, the at least one first lateral dimension is a plurality of lateral dimensions, wherein each of the plurality of lateral dimensions are greater than the at least one second lateral dimension.

According to the first aspect of the present disclosure, the at least one first lateral dimension includes a plurality of lateral dimensions, wherein the plurality of lateral dimensions decrease in magnitude from the bottom surface of the bottom portion toward a top surface of the bottom portion.

According to the first aspect of the present disclosure, the augmentation component includes an interior volume configured to receive a protrusion disposed on a surface of the tibial component.

According to the first aspect of the present disclosure, the instrument is a reaming instrument configured to ream a volume that corresponds to a volume of the augmentation component.

According to the first aspect of the present disclosure, the instrument includes a handle and an engagement component. The engagement component is coupled with the handle and includes a lower engagement portion having a plurality of first engagement features disposed on at least a portion of at least a lateral surface and a top surface of the lower engagement portion, and an upper engagement portion positioned superior to the lower engagement portion that includes a plurality of second engagement features disposed on at least a portion of at least a lateral surface and a top surface of the upper engagement portion.

According to the first aspect of the present disclosure, the plurality of first engagement features and the plurality of second engagement features protrude from the surfaces of the lower engagement portion and the upper engagement portion.

A second aspect of the present disclosure is an augmentation component for a tibial component. The augmentation component includes a top portion with at least one lateral dimension, and a bottom portion with a plurality of lateral dimensions that decrease in magnitude from a bottom surface of the bottom portion to a top surface of the bottom portion.

According to the second aspect of the present disclosure, the top portion is positioned superior to and is integral with the bottom component.

According to the second aspect of the present disclosure, augmentation component also includes a central volume extending from the bottom surface of the bottom portion into the augmentation component, the central volume having an interior surface.

According to the second aspect of the present disclosure, the augmentation component also includes a bore extending from an exterior surface of the augmentation component through to the interior surface of the augmentation component and providing fluid communication from the central volume to the exterior surface of the augmentation component.

According to the second aspect of the present disclosure, the augmentation component also includes a first porous structure having a first porosity and a second porous structure having a second porosity, wherein the first porous structure is positioned proud relative to the second porous structure.

According to the second aspect of the present disclosure, the first porosity is greater than the second porosity.

According to the second aspect of the present disclosure, the second engagement element includes a retention portion comprising a post and a threading, wherein the post is configured to releasably couple with the second arm and the threading is configured to releasably couple with the actuator.

A third aspect of the present disclosure is a method of augmenting a tibial component. The method includes collecting imaging data from a patient, identifying from the imaging date a void in a distal portion of a tibia of the patient, obtaining an augmentation component with a volume that corresponds to a volume of the void of the patient, coupling the augmentation component with a tibial component, and implanting the tibial component such that the augmentation component occupies at least a portion of the void of the patient.

### BRIEF DESCRIPTION OF THE DRAWINGS

The accompanying drawings, which are incorporated in and constitute a part of the specification, illustrate embodiments of the inventions and together with the detailed description herein, serve to explain the principles of the inventions. It is emphasized that, in accordance with the standard practice in the industry, various features may or may not be drawn to scale. In fact, the dimensions of the various features may be arbitrarily increased or reduced for clarity of discussion. The drawings are only for purposes of illustrating embodiments of inventions of the disclosure and are not to be construed as limiting the inventions.

FIG. 1 is a front-right perspective view of an exemplary augmented system for ankle arthroplasty procedures, in accordance with the present disclosure;

FIG. 2 is a front view of the exemplary augmented system for the ankle arthroplasty procedures of FIG. 1, in accordance with the present disclosure;

FIG. 3 is front view of a portion of the exemplary augmented system for the ankle arthroplasty of FIG. 1, in accordance with the present disclosure;

FIG. 4 is a bottom view of the portion of FIG. 3 of the exemplary augmented system for the ankle arthroplasty of FIG. 1, in accordance with the present disclosure;

FIG. 5A is a rear-left perspective view of an instrument to be implemented in conjunction with the exemplary system for performing the ankle arthroplasty of FIG. 1, in accordance with the present disclosure;

FIG. 5B is side cross-sectional view of an accessory for the instrument of FIG. 5A to be implemented in conjunction with the exemplary system for performing the ankle arthroplasty of FIG. 1, in accordance with the present disclosure;

FIG. 6 is a top view of the instrument of FIG. 5A to be implemented in conjunction with the exemplary system of FIG. 1 for implementation in performing a surgical procedure, in accordance with the present disclosure;

FIG. 7 is a front-right perspective view of the instrument of FIG. 5A to be implemented in conjunction with the exemplary system of FIG. 1 for implementation in performing a surgical procedure, in accordance with the present disclosure;

FIG. 8 is a top view of a portion of the instrument of FIG. 5A to be implemented in conjunction with the exemplary system of FIG. 1 for implementation in performing a surgical procedure, in accordance with the present disclosure;

FIG. 9 is a front view of a portion of the instrument of FIG. 5A to be implemented in conjunction with the exemplary system of FIG. 1 for implementation in performing a surgical procedure, in accordance with the present disclosure;

FIG. 10 is a front perspective view of an exemplary augmented system for ankle arthroplasty procedures, in accordance with the present disclosure;

FIG. 11 is a perspective front-left view of the exemplary augmented system of FIG. 10, in accordance with the present disclosure;

FIG. 12 is front exploded view of the exemplary augmented system of FIG. 10, in accordance with the present disclosure;

FIG. 13 is top view of the exemplary augmented system of FIG. 10, in accordance with the present disclosure;

FIG. 14 is a front perspective view of a portion of the exemplary augmented system of FIG. 10, in accordance with the present disclosure;

FIG. 15 is a top view of the portion of the exemplary augmented system of FIG. 10, in accordance with the present disclosure;

FIG. 16 is a front view of the anatomy of the distal tibia and fibula prior to placing a tibial component of a total ankle arthroplasty system, in accordance with the present disclosure;

FIG. 17 is a front view of the anatomy of FIG. 16 shown adjacent an exemplary instrument for implementation with the augmented system of FIG. 10, in accordance with the present disclosure;

FIG. 18 is a front view of the anatomy of FIG. 16 after implementation of the exemplary instrument of FIG. 17, in accordance with the present disclosure;

FIG. 19 is a front view of the anatomy of FIG. 16 after implementation of the exemplary instrument of FIG. 17 and adjacent the augmented system of FIG. 10, in accordance with the present disclosure;

FIG. 20 is a front view of the anatomy of FIG. 16 after implementation of the exemplary instrument of FIG. 17 and the implantation of the augmented system of FIG. 10, in accordance with the present disclosure;

FIG. 21 is a front-left perspective view of an implant system for a lower extremity, in accordance with the present disclosure;

FIG. 22 is an alternate front-left perspective view of the implant system of FIG. 21 to be implemented in conjunction with the exemplary system of FIG. 1, in accordance with the present disclosure;

FIG. 23 is a front view of a portion of the implant system of FIG. 21, in accordance with the present disclosure;

FIG. 24 is a side view of the portion of FIG. 23 of the implant system of FIG. 21 in accordance with the present disclosure;

FIG. 25 is a front-left perspective view of the portion of FIG. 23 of the implant system of FIG. 21, in accordance with the present disclosure;

FIG. 26 is an alternate front-left perspective view of the portion of FIG. 23 of the implant system of FIG. 21 implanted in the distal tibia, in accordance with the present disclosure;

FIG. 27 is a front view of an instrument which may be implemented in conjunction with the implant system of FIG. 21, in accordance with the present disclosure;

FIG. 28 is a front-left perspective view of the instrument of FIG. 27 which may be implemented in conjunction with the implant system of FIG. 21, in accordance with the present disclosure;

FIG. 29 is a top view of an instrument which may be implemented in conjunction with the implant system of FIG. 21, in accordance with the present disclosure;

FIG. 30 is a front-left perspective view of the instrument of FIG. 29 which may be implemented in conjunction with the implant system of FIG. 21, in accordance with the present disclosure;

FIG. 31 is a side view of an instrument which may be implemented in conjunction with the implant system of FIG. 21, in accordance with the present disclosure;

FIG. 32 is a front view of the instrument of FIG. 31 which may be implemented in conjunction with the implant system of FIG. 21, in accordance with the present disclosure;

FIG. 33 is a top view of the instrument of FIG. 31 which may be implemented in conjunction with the implant system of FIG. 21, in accordance with the present disclosure;

FIG. 34 is a side view of the instrument of FIG. 31 implemented in conjunction with other instruments which may be implemented in conjunction with the implant system of FIG. 21, in accordance with the present disclosure;

FIG. 35 is a side view of an instrument which may be implemented in conjunction with the implant system of FIG. 21, in accordance with the present disclosure;

FIG. 36 is a top view of the instrument of FIG. 35 which may be implemented in conjunction with the implant system of FIG. 21, in accordance with the present disclosure;

FIG. 37 is a front-right perspective view of the instrument of FIG. 35 which may be implemented in conjunction with the implant system of FIG. 21, in accordance with the present disclosure;

FIG. 38 is a front-left perspective view of the instruments of FIGS. 29 and 35 implemented in conjunction with one another, which may also be implemented in conjunction with the implant system of claim 21, in accordance with the present disclosure;

FIG. 39 is a front view of the implant system of FIG. 21, in accordance with the present disclosure;

FIG. 40 is a rear view of the implant system of FIG. 21, in accordance with the present disclosure;

FIG. 41 is a side view of the implant system of FIG. 21, in accordance with the present disclosure,

FIG. 42 is perspective view of the portion of the exemplary augmented system of FIG. 3 uncoupled from a tibial component, in accordance with the present disclosure;

FIG. 43 is a perspective view of the portion of the exemplary augmented system of FIG. 3 being coupled to the tibial component of FIG. 42, in accordance with the present disclosure;

FIG. 44 is a top view of the assembly process of the augmented system of FIG. 1, in accordance with the present disclosure;

FIG. 45 is a top view of the coupling process for the augmented system of FIG. 1, in accordance with the present disclosure;

FIG. 46 is a side view of the assembled augmented system of FIG. 1, in accordance with the present disclosure; and

FIG. 47 is a front view of the assembled augmented system of FIG. 1 implanted into a distal tibia portion, in accordance with the present disclosure.

### DETAILED DESCRIPTION FOR CARRYING OUT THE INVENTION

In this detailed description and the following claims, the words proximal, distal, anterior or plantar, posterior or dorsal, medial, lateral, superior and inferior are defined by their standard usage for indicating a particular part or portion of a bone or implant according to the relative disposition of the natural bone or directional terms of reference. For example, “proximal” means the portion of a device or implant nearest the torso, while “distal” indicates the portion of the device or implant farthest from the torso. As for directional terms, “anterior” is a direction towards the front side of the body, “posterior” means a direction towards the back side of the body, “medial” means towards the midline of the body, “lateral” is a direction towards the sides or away from the midline of the body, “superior” means a direction above and “inferior” means a direction below another object or structure. Further, specifically in regards to the foot, the term “dorsal” refers to the top of the foot and the term “plantar” refers the bottom of the foot.

Similarly, positions or directions may be used herein with reference to anatomical structures or surfaces. For example, as the current implants, devices, instrumentation, and methods are described herein with reference to use with the bones of the foot, the bones of the foot, ankle and lower leg may be used to describe the surfaces, positions, directions or orientations of the implants, devices, instrumentation and methods. Further, the implants, devices, instrumentation, and methods, and the aspects, components, features and the like thereof, disclosed herein are described with respect to one side of the body for brevity purposes. However, as the human body is relatively symmetrical or mirrored about a line of symmetry (midline), it is hereby expressly contemplated that the implants, devices, instrumentation, and methods, and the aspects, components, features and the like thereof, described and/or illustrated herein may be changed, varied, modified, reconfigured or otherwise altered for use or association with another side of the body for a same or similar purpose without departing from the spirit and scope of the invention. For example, the implants, devices, instrumentation, and methods, and the aspects, components, features and the like thereof, described herein with respect to the right foot may be mirrored so that they likewise function with the left foot. Further, the implants, devices, instrumentation, and methods, and the aspects, components, features and the like thereof, disclosed herein are described with respect to the foot for brevity purposes, but it should be understood that the implants, devices, instrumentation, and methods may be used with other bones of the body having similar structures.

Surgical procedures are commonly performed to address acute or chronic conditions of a patient that can be addressed by placing an implant (e.g., plate, screw, component of an implant system, etc.) adjacent (e.g., coupled with, abutting, etc.). These procedures can include arthroplasty procedures (e.g., joint replacement) such as an ankle arthroplasty procedure which is used herein as an exemplary arthroplasty procedure and an exemplary application of various implants, systems, augmentation components, and methodologies. Accordingly, the application of an ankle arthroplasty procedure should not be considered limiting but rather exemplary as the contents of this disclosure may be applied to alternate implants, implant systems, and procedures/applications.

Ankle arthroplasty implants commonly include at least tibial and talar components which couple with the distal tibia and proximal talus, typically after at least some resection of said bones has occurred. For the sake of this disclosure, the tibial component of ankle arthroplasty systems will be discussed and contemplated relative to the systems, components, and methods disclosed herein. However, it should be noted that said systems, components, and methods may also be applicable to the talar component of various ankle arthroplasty systems (or other components of arthroplasty systems, ankle or otherwise).

The tibial component of ankle arthroplasty systems (including that which is shown and described subsequently herein) is typically impacted into a recess formed in the distal tibia after at least a portion of the distal tibia has been resected. In some procedures and for some patients, the distal tibia is healthy and accommodates this resection and impaction of the tibial component with sufficient amounts of the distal tibia to provide bone purchase and ingrowth, thus retaining the tibial component within the desired position in the distal tibia. However, some patients exhibit one or more of various irregularities in the distal tibia, some of which may be identified using preoperative imaging (e.g., CT, MRI, etc.) while others may become apparent intraoperatively. These irregularities may include cysts, voids, and other volumes within the distal tibia that prevent the tibial component from achieving proper integration with the distal tibia, as there is not a sufficient amount of healthy distal tibia to provide the necessary bone ingrowth surfaces and bone purchase to retain the tibial component. Standard tibial components are typically configured for placement in a healthy distal tibia that is absent any irregularities. However, when irregularities become evident (or other anatomical challenges, such as ankle arthroplasty revision procedures standard tibial components fail to accommodate the anatomy of the distal tibia of patients.

Accordingly, it is desirable to tailor a standard tibial component to patients with irregularities in the distal tibia. In some instances, custom implants can address irregularities but can be prohibitively expensive and carry lengthy lead times. Additionally, custom implants cannot account for irregularities that are identified intraoperatively. In order to adapt standard tibial components for various irregularities in the distal tibia, an augment is required that may couple with the superior (top) surface of the tibial component and occupy at least a portion of such irregularities in the distal tibia, thus allowing the standard tibial component to occupy the resected volume and contact appropriate surfaces of the distal tibia. Such an augment would aid in fixation of the tibial component by providing fixation (via bone ingrowth surfaces, bone purchase, etc.) within said irregularities of the distal tibia and accordingly adapt standard tibial components for patients presenting distal tibial irregularities.

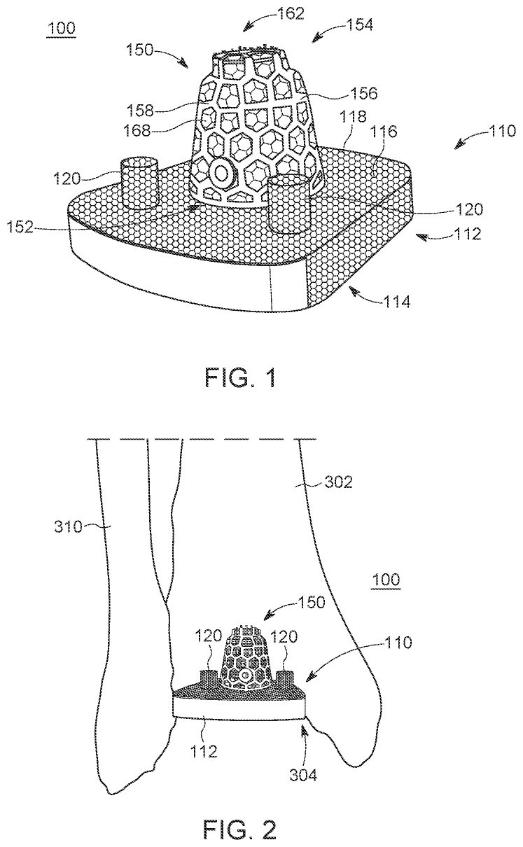

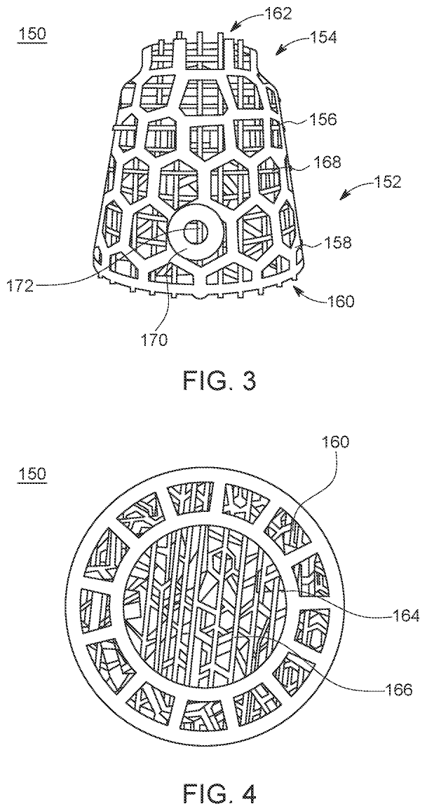

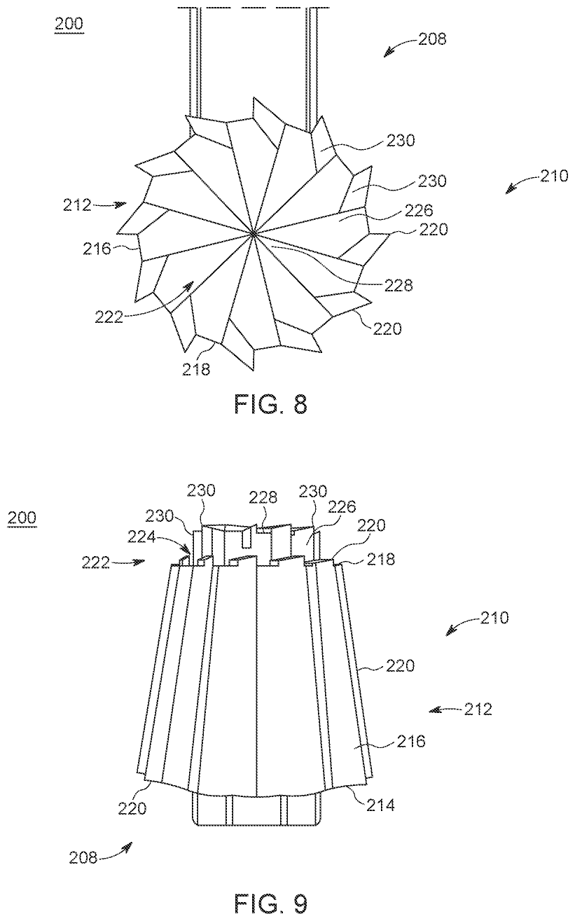

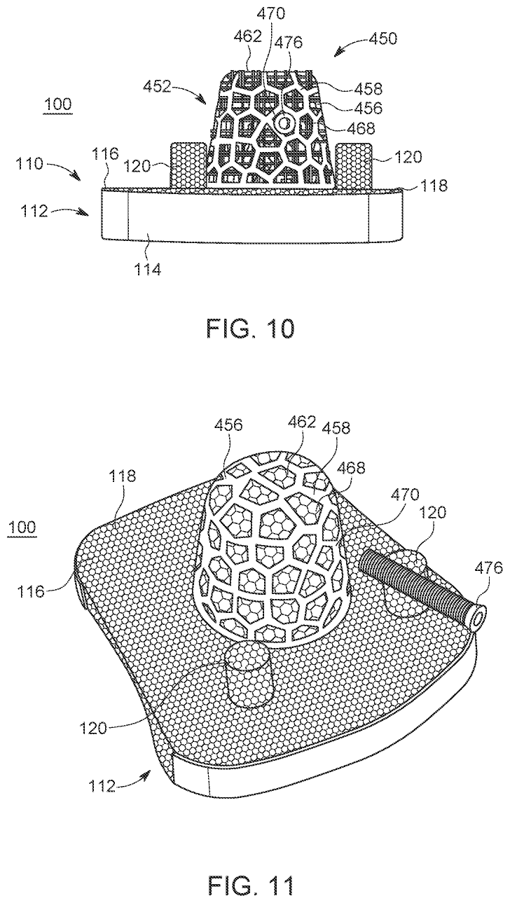

Referring to the drawings, wherein like reference numerals are used to indicate like or analogous components throughout the several views, and with particular reference to FIGS. 1-9, there is illustrated an exemplary embodiment of an implant system 100 which may be a portion of a larger implant system (for example, an arthroplasty system). As shown, the implant system 100 is a tibial component system which is a portion of a total ankle arthroplasty system, where the implant system 100 is coupled with the distal tibia after a portion of the distal tibia has been resected. The implant system 100 is shown to include a tibial component 110 having a base portion 112 as well as bottom and top surfaces 114 and 116, respectively, where the top surface 114 is arranged opposite the base portion 112 from the bottom surface 116. The tibial component 110 is shown to include a texture 118 on the outer surface of the base portion 112 as well as projections 120 extending upward from the top surface 116 of the base portion 112. In some embodiments, the tibial component 110 may include an alternate number of projections 120, for example one protrusion, three protrusions, etc. As shown, the projections 120 are configured to facilitate coupling with the distal tibia after resection as occurred, with said projections 120 extending into corresponding openings in the tibia created by a physician in the process of preparing the distal tibia for the tibial component 110. The texture 118 may be configured to facilitate bone ingrowth and/or otherwise retain the tibial component in the distal tibia. It should be noted that the tibial component 110 is a standard tibial component (e.g., has no custom components, is sold off the shelf, etc.) and is shown here for exemplary purposes.

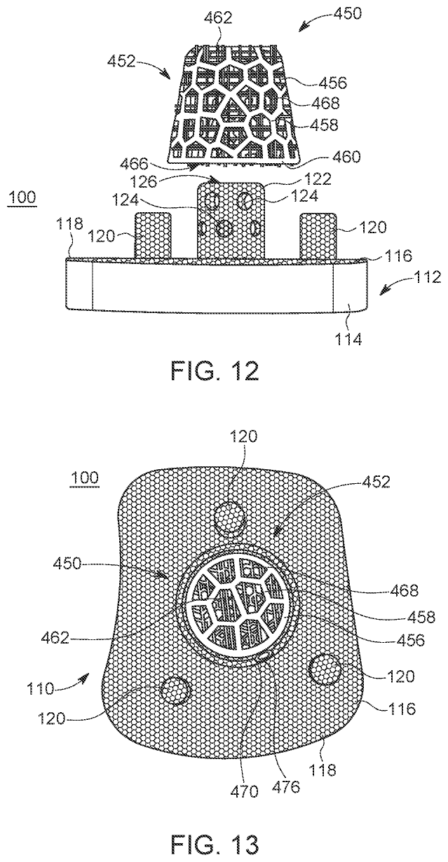

The tibial component 110 further includes a stem 122 (as shown in FIG. 12) arranged in a central portion of the top surface 116, with the stem 122 extending perpendicularly upward from the top surface 116 (similar to the projections 120). The stem 122 is configured to extend upward (e.g., proximally, in a superior direction) into the distal tibia when placed in a similar fashion to the projections 122, where one or more instruments creates a volume in the distal tibia of which the stem 122 occupies at least a portion. The stem 122 as shown includes a substantially cylindrical geometry, where a bottom end of the stem is integral with the top surface 116 (as are the projections 120) and a top end of the stem 122 is open. Accordingly, the stem 122 includes a central opening 126 (e.g., a volume with the bottom of said volume defined by the top surface 116) with a complimentary cylindrical geometry to that of the stem 122. The stem 122 is further shown to include apertures 124 arranged about the lateral surface of the stem 122. As shown, each of the apertures 124 are substantially the same size, but in some aspects one or more of said apertures 124 may be of an alternate size (e.g., to accommodate another component, etc.). Similarly, the apertures 124 are all of a substantially circular geometry but may have alternate geometries in some embodiments. The stem 122 also includes the texture 118 on all surfaces thereof, according to the exemplary embodiments shown and described herein.

An augment 150 is shown to be coupled with the top surface 116 of the tibial component 110. With reference to FIG. 2, the augment 150 includes a central opening 166 that is sized to receive at least a portion of the stem 122 therein when the augment 150 is coupled with the tibial component. In some aspects, the augment 150 may be coupled with the tibial component via an adhesive or cement, or may implement other coupling methods (e.g., fasteners, etc.). As shown, the tibial component 110 includes a single augment 150, but in some embodiments multiple augments 150 of various geometries and/or sizes may be coupled with the tibial component 110. The augment 150 is shown to have a height greater than that of the stem 122 such that when implanted in the distal tibia of a patient, the augment 150 extends further upward (in a proximal, superior direction) than the stem 122 or the projections 120. The augment 150 may be coupled with the tibial component (e.g., positioned on the top surface 116) so as to provide a volume configured to occupy at least a portion of a complimentary volume of an irregularity in the distal tibia of the patient. Further, in some aspects the augment 150 may be configured to occupy the entirety of the volume of an irregularity or may be sized so as to have a volume greater than said irregularity (e.g., to facilitate a desired fit). Furthermore, and as shown and described subsequently herein, the augment 150 may include one or more complimentary instruments configured to facilitate implantation of the tibial component 110 with the augment 150 (e.g., a reamer to ream an irregularity into a volume that will receive the augment 150, etc.). In some aspects, the augment may be of a custom size based on an irregularity identified and measured via imaging data. Further, in some aspects, the augment 150 may be selected by a physician from a library of augments 150 (and/or augment trials) provided in a surgical kit, where each augment 150 has a different size/shape/geometry so as to address various irregularities in the distal tibia of the patient. It should be noted that an appropriate version of the augment 150 for an irregularity (or potentially multiple irregularities) may require manipulation of the geometry of the irregularity in order for the augment 150 to fit as desired.

As shown, the augment 150 has a bottom portion 152 and a top portion 154. The geometry of the bottom portion 152 of the augment 150 is that of a frustum of a cone shape (e.g., where a frustum is a portion of a solid that lies between two parallel lines cutting said solid), and the geometry of the top portion 154 is that of a cylinder with a diameter greater than the height. The cylindrical geometry of the top portion 154, as described subsequently herein, corresponds to a complimentary cylindrical volume formed in the distal portion of the tibia prior to implantation. Such cylindrical geometry (both of the top portion 154 and said corresponding volume) is configured to facilitate coupling of the augment 150 (and other components of the system as shown and described) with the distal tibia and retention thereof. It should be noted that in some aspects the geometry of the augment 150 may be variable (e.g., the bottom portion 152 may have a lesser height/volume/surface area than that of the top portion 154, the bottom portion 152 and top portion 154 may each have alternate geometries, etc.).

The augment 150 further includes an exterior surface 156, a bottom surface 160, a top surface 162, and an interior surface 164. The exterior surface 156 extends between the bottom surface 160 and the top surface 162. As shown, the bottom surface 160 is coupled with the top surface 116 of the tibial component 110. The top surface 162 is arranged opposite the augment 150 from the bottom surface and is an upper surface of the top portion 154 of the augment 150. The interior surface 164, as shown in FIG. 4, defines the volume of the central opening 166 of the augment 150. The augment 150 also has a pair of porous structures (e.g., lattice structures, web structures, nodal structures, mesh structures, etc.) including a first porous structure 158 and a second porous structure 168. As shown, the first porous structure 158 is arranged proud relative to the second porous structure 168, where the first porous structure 158 defines an outer surface of the augment 150 for all of the aforementioned surfaces. The first porous structure 158 is shown to have a greater porosity (e.g., more porous) than that of the second porous structure 168. As shown, the first porous structure is a substantially randomized structure (while still satisfying various design inputs and structural parameters) whereas the second porous structure has a grid-shaped geometry (e.g., with substantially orthogonal components). It should be understood that the first and second porous structures 158, 168 as shown and described herein are exemplary and may be modified for various embodiments of the augment 150. For example, a particular irregularity in the distal tibia of a patient may be conducive to one or more porous structures with greater or lesser porosities than those shown in the exemplary augment 150. Ultimately, the augment 150 may include an alternate number of porous structures or alternate porous structures, or an alternate arrangement of two or more porous structures.

The augment 150 includes an exterior opening 170 disposed on the exterior surface 156 thereof as well as an interior opening 174 disposed on the interior surface 164 thereof. The exterior opening 170 and the interior opening 174 define a bore 172 extending from the exterior surface 156 to the interior surface 164 and thus establishing fluid communication between said surfaces (and between the exterior of the augment 150 and the central opening 166). In some aspects, the augment 150 may include the bore 172 to provide optionality for a physician to provide additional fixation when coupling the augment 150 with the tibial component 110. For example, in addition to implementing an adhesive/cement to couple the bottom surface 160 of the augment 150 with the top surface 116 of the tibial component the physician may implement a fastener (e.g., screw, etc.). Such a fastener may be inserted through the exterior opening 170, into and through the bore 172 (e.g., the bore receives the fastener), and exit the interior opening 174. The fastener, and thus the bore 172, may be aligned with one or more of the apertures 124 of the stem 122 such that one or more of the apertures 124 receive at least a portion of the fastener thus providing additional coupling between the augment 150 and the tibial component 110.

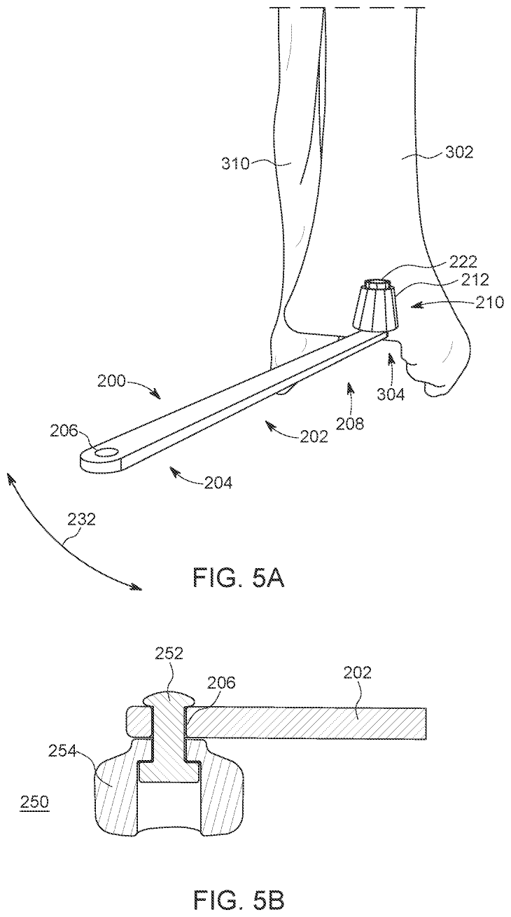

Referring now to FIGS. 5A-9, an instrument 200 is shown (and with reference to FIG. 5, relative to an exemplary tibia 302 and fibula 310 of a patient). The instrument 200 is shown to include a handle 202 having a first end 204 and a second end 208 which are disposed at opposite ends of the handle 202 as shown in the exemplary embodiment of FIGS. 5-9. The first end 204 of the handle 202 includes an opening 206 extending through the handle 202. The handle 200 also includes an engagement portion 210 arranged at (or adjacent to) the second end 208 of the handle 200, where the engagement portion 210 is coupled with the second end 208. In some aspects, the engagement portion 210 may be coupled with the second end 208 through a variety of means (e.g., pivotably coupled, rotatably coupled, etc.). Further, in some aspects the engagement portion 210 may be at least partially integral with the second end 208 of the handle 200.

Referring now to FIG. 5B, an accessory 250 is shown that may be coupled with the opening 206 disposed at the second end 208 of the handle 202. The accessory 250 is shown to include a stem 252 configured to facilitate rotatable and/or pivotable coupling with the handle 202 via the opening 206. The stem 252 may include one or more geometries and/or dimensions, where at least one of said geometries is a shape complimentary to that of the opening 206. For example, as shown the stem 252 include a substantially cylindrical portion having a lateral dimension lesser than that of the opening 206 (e.g., a diameter) such that said cylindrical portion may be at least partially received by and/or disposed within the opening 206. Further, the stem 252 is shown to include geometries at opposite ends of the cylindrical portion with a lateral dimension greater than that of the opening 206 so as to retain at least a portion of the cylindrical portion within the opening 206 (and thus retain the accessory 150 in a coupled state with the handle 202). The accessory 250 is further shown to include a knob 254, where the knob 254 is rotatably and/or pivotably coupled with the stem 252. As shown, the knob 254 includes a central opening configured to engage one of the aforementioned geometries of the stem 252 that has a first lateral dimension greater than that of the cylindrical portion of the stem 252. The central opening of the knob 254 is also shown to have a second lateral dimension where said lateral dimension is greater than that of the cylindrical portion of the stem 252, but lesser than that of the end geometry of the stem 252 that is greater than that of the cylindrical portion (e.g., said second opening of the knob 254 may have a lateral dimension the same as or similar to that of the opening 206). Accordingly, the knob 252 may be rotated about the stem 252 independent of any movement to the handle 202 of the instrument 200 and, as described subsequently herein, facilitate other manipulation of the instrument 200.

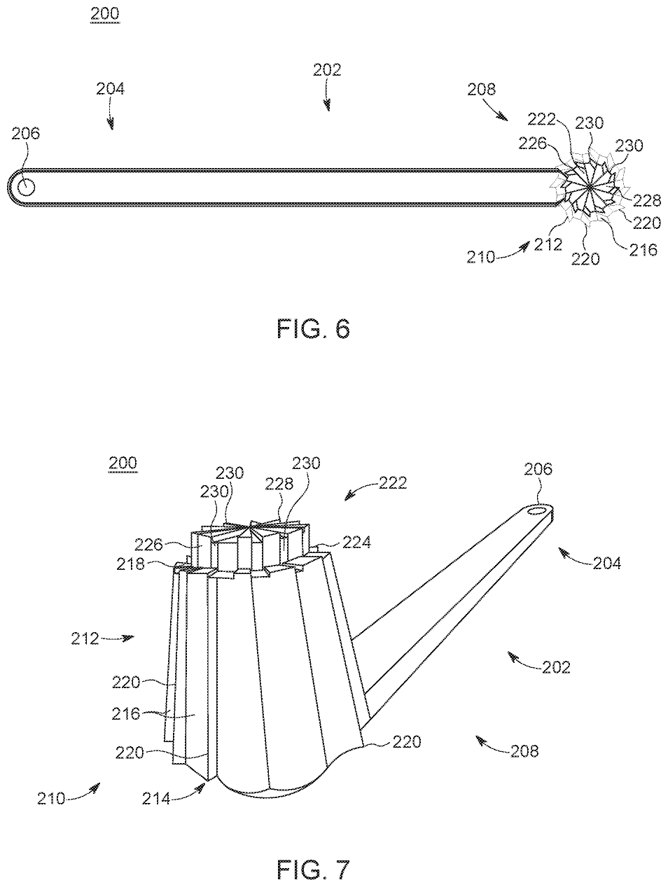

The engagement portion 210 is shown to include a first portion 212 and second portion 222 (e.g., bottom and top portions, respectively) where the first portion 212 and the second portion 222 are integral with one another. The first portion 212 includes a bottom surface 214 and a top surface 218, as well as a lateral surface 216 extending between the bottom surface 214 and the top surface 218. The lateral surface 216 includes a plurality of first engagement features 220 extending (e.g., projecting, protruding, etc.) from the lateral surface 216 at oblique and/or orthogonal angles relative to the lateral surface 216. Further, as shown the first engagement features 220 are disposed substantially equidistant one another and extend vertically along the lateral surface 216 from the bottom surface 214 to the top surface 218 of the first portion 212. In some aspects, the first engagement features 220 may extend vertically beyond the lateral surface 216 (e.g., past the bottom surface 214 and the top surface 218). Each of the first engagement features 220 as shown includes a ridge protruding from the lateral surface 216 of the first portion 212, with said ridges each angled in a specific direction (e.g., as shown in FIG. 6, the first engagement features 220 are angled in a substantially counterclockwise direction relative to the engagement portion 210). The first portion 212 is configured to have a geometry similar to that of the bottom portion 152 of the augment 150 such that implementation of the instrument 200 (and the first portion 212) may ream or otherwise configured a space in the distal tibia 302 of the patient to accommodate at least a portion of the augment 150. Accordingly, the geometry of the first portion 212 of the engagement portion 210 has the general geometry of the frustum of a cone as defined previously herein with reference to the augment.

The second portion 222 of engagement portion 210 includes a bottom surface 224 and a top surface 228, as well as a lateral surface 226 extending between the bottom surface 224 and the top surface 228. As shown, the second portion 222 is arranged such that the bottom surface 224 abuts (e.g., is integral with) the top surface 220 of the first portion 212. The second portion 222 is shown to have a substantially cylindrical geometry (similar to that of the top portion 154 of the augment 150), with the diameter of the cylinder greater than the height (although all geometric properties of the instrument 200 may vary based on the geometry of the augment 150). Said geometry of the second portion 222 is configured to ream a volume in the distal tibia complimentary to the geometry of the top portion 164 of the augment 150 (e.g., to ream a cylindrical volume). The second portion 222 is shown to include a plurality of second engagement features 230, which may be arranged the same and/or similar to those of the first portion 212. As shown, the second engagement features 230 extend laterally (e.g., project, protrude, etc.) from the lateral surface 226 and further extend vertically along at least the height of the lateral surface 226. As shown, the second engagement features 230 extend from the bottom surface 224 of the second portion 222 vertically and terminate just beyond the top surface 228 such that that top surface 228 includes the second engagement features 230. It should be understood that the augment 150 may be configured such that it may be 3-D printed.

Referring to FIG. 5, the instrument 200 is shown to be engaged with the exemplary tibia 302 of a patient. The engagement portion 210 has been placed such that the top surface 228 contacts a distal surface of a recess 304 created in the distal tibia 302 (e.g., by resection). In the instance of a revision procedure, the engagement portion 210 may be placed in a portion of the distal tibia 302 which previously accommodated the stem 122 of the tibial component 110. In such an instance, the geometry of the engagement portion 210 allows for the engagement portion 210 to self-center when reaming a volume to accommodate the augment 150. That is to say that the geometry of the engagement portion 210 of the instrument 200 is configured to ream a volume centered about (and in this example, extending concentrically therefrom) the recess in the distal tibia that previously received the stem 122 (or other protrusions of other tibial components). In procedures that do not offer such a recess from a previous tibial component, the engagement portion is configured to center about a point identified by the physician and ultimately ream a volume with a geometry complimentary to that of the augment 150 such that said volume will receive the augment 150 (or at least a portion thereof) when implanted after coupling of the augment 150 with the tibial component 110. The instrument 200 as shown, is a manual use instrument where a physician may manipulate the instrument 200 by the handle 202 about a semi-circular path 232 in a reciprocating nature to ream the desired volume in the distal tibia 302. Movement about the path 232 may be driven by a physician manipulating the instrument 200 via the accessory 250 as shown in FIG. 5B. For example, the instrument 200 may be manipulated in the semi-circular path 232 by grasping the knob 254, where the knob rotates to facilitate such manipulation and movement of the instrument 200. In some aspects, the instrument 200 and/or portions thereof may be modified so as to couple with various other instruments (e.g., power tools/reamers). The instrument 200 may also be configured/designed such that it may be 3-D printed, thus eliminating the machining process common to many instruments.

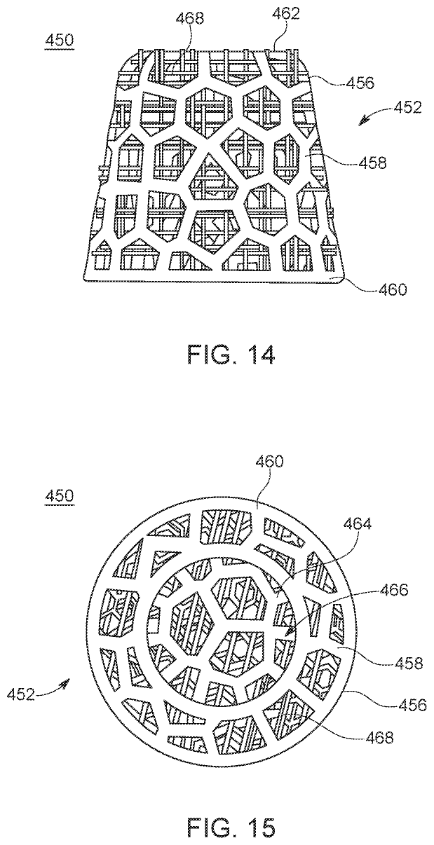

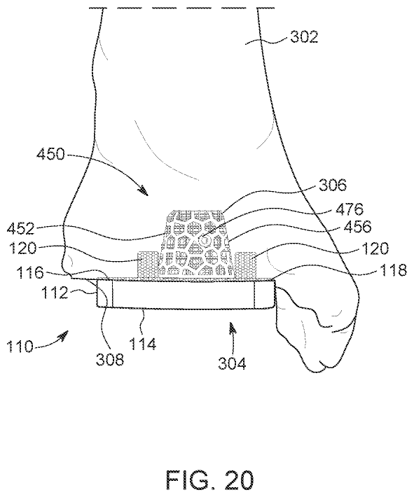

Referring now to FIGS. 11-20, alternate embodiments of an augment 450 and an instrument 500 are shown. The augment 450 may have one or more properties the same as and/or similar to the augment 150 and, similarly, the instrument 500 may have one or more properties the same as the instrument 200. As shown in FIGS. 11-20, the augment 450 is shown relative to the tibial component system 100 and components thereof that are the same as those shown and described previously herein. For the sake of brevity, these components will not be described again. However, it should be understood that similar to the augment 150 and instrument 200, the augment 450 and the instrument 500 may be implemented in conjunction with various implant systems (tibial and otherwise) and the tibial component system 100 is shown herein as an exemplary tibial component. It should also be understood that the applications for the augment 450 and instrument 500 include at least those shown and described previously here (e.g., to address one or more irregularities in the anatomy of a patient, whether preoperatively through custom design and/or intraoperatively through a trialing process).

The augment 450 is shown to be coupled with the top surface 116 of the tibial component 110. With reference to FIG. 15, the augment 450 includes a central opening 466 that is sized to receive at least a portion of the stem 122 therein when the augment 450 is coupled with the tibial component. In some aspects, the augment 450 may be coupled with the tibial component via an adhesive or cement, or may implement other coupling methods (e.g., fasteners, etc.). As shown, the tibial component 110 includes a single augment 450, but in some embodiments multiple augments 450 of various geometries and/or sizes may be coupled with the tibial component 110. The augment 450 is shown to have a height greater than that of the stem 122 such that when implanted in the distal tibia of a patient, the augment 450 extends further upward (in a proximal, superior direction) than the stem 122 or the projections 120. The augment 450 may be coupled with the tibial component (e.g., positioned on the top surface 116) so as to provide a volume configured to occupy at least a portion of a complimentary volume of an irregularity in the distal tibia of the patient. Further, in some aspects the augment 450 may be configured to occupy the entirety of the volume of an irregularity or may be sized so as to have a volume greater than said irregularity (e.g., to facilitate a desired fit). Furthermore, and as shown and described subsequently herein, the augment 450 may include one or more complimentary instruments configured to facilitate implantation of the tibial component 110 with the augment 450 (e.g., a reamer to ream an irregularity into a volume that will receive the augment 450, etc.). In some aspects, the augment 450 may be of a custom size based on an irregularity identified and measured via imaging data. Further, in some aspects, the augment 450 may be selected by a physician from a library of augments 450 (and/or augment trials) provided in a surgical kit, where each augment 450 has a different size/shape/geometry so as to address various irregularities in the distal tibia of the patient. It should be noted that an appropriate version of the augment 450 for an irregularity (or potentially multiple irregularities) may require manipulation of the geometry of the irregularity in order for the augment 450 to fit as desired.

As shown, the augment 450 has a body 452, with the geometry of the body 452 of the augment 450 is that of a frustum of a cone shape (e.g., where a frustum is a portion of a solid that lies between two parallel lines cutting said solid. It should be noted that in some aspects the geometry of the augment 450 may be variable (e.g., the body 452 may have a diameter greater or less than the height of the body 452, the body 452 may have alternate geometries, etc.). The augment 450 further includes an exterior surface 456, a bottom surface 460, a top surface 462, and an interior surface 464. The exterior surface 456 extends between the bottom surface 460 and the top surface 462. As shown, the bottom surface 460 is coupled with the top surface 116 of the tibial component 110. The top surface 462 is arranged opposite the augment 450 from the bottom surface and is an upper surface of the body 454 of the augment 450. The interior surface 464, as shown in FIG. 15, defines the volume of the central opening 466 of the augment 450. The augment 450 also has a pair of porous structures (e.g., lattice structures, web structures, nodal structures, mesh structures, etc.) including a first porous structure 458 and a second porous structure 468. As shown, the first porous structure 458 is arranged proud (e.g., is the outer structure relative to an inner structure) relative to the second porous structure 468, where the first porous structure 458 defines an outer surface of the augment 450 for all of the aforementioned surfaces. The first porous structure 458 is shown to have a greater porosity (e.g., more porous) than that of the second porous structure 468. As shown, the first porous structure 458 is a substantially randomized structure (while still satisfying various design inputs and structural parameters) whereas the second porous structure has a grid-shaped geometry (e.g., with substantially orthogonal components). It should be understood that the first and second porous structures 458, 468 as shown and described herein are exemplary and may be modified for various embodiments of the augment 450. For example, a particular irregularity in the distal tibia of a patient may be conducive to one or more porous structures with greater or lesser porosities than those shown in the exemplary augment 450. Ultimately, the augment 450 may include an alternate number of porous structures or alternate porous structures, or an alternate arrangement of two or more porous structures.

The augment 450 includes an exterior opening 470 disposed on the exterior surface 456 thereof as well as an interior opening 474 disposed on the interior surface 464 thereof. The exterior opening 470 and the interior opening 474 define a bore 472 extending from the exterior surface 456 to the interior surface 464 and thus establishing fluid communication between said surfaces (and between the exterior of the augment 450 and the central opening 466). In some aspects, the augment 450 may include the bore 472 to provide optionality for a physician to provide additional fixation when coupling the augment 450 with the tibial component 110. For example, in addition to implementing an adhesive/cement to couple the bottom surface 460 of the augment 450 with the top surface 116 of the tibial component the physician may implement a fastener 476 (e.g., screw, etc.). The fastener 476 may be inserted through the exterior opening 470, into and through the bore 472 (e.g., the bore receives the fastener), and exit the interior opening 474. The fastener 476, and thus the bore 472, may be aligned with one or more of the apertures 124 of the stem 122 such that one or more of the apertures 124 receive at least a portion of the fastener 476 thus providing additional coupling between the augment 450 and the tibial component 110.

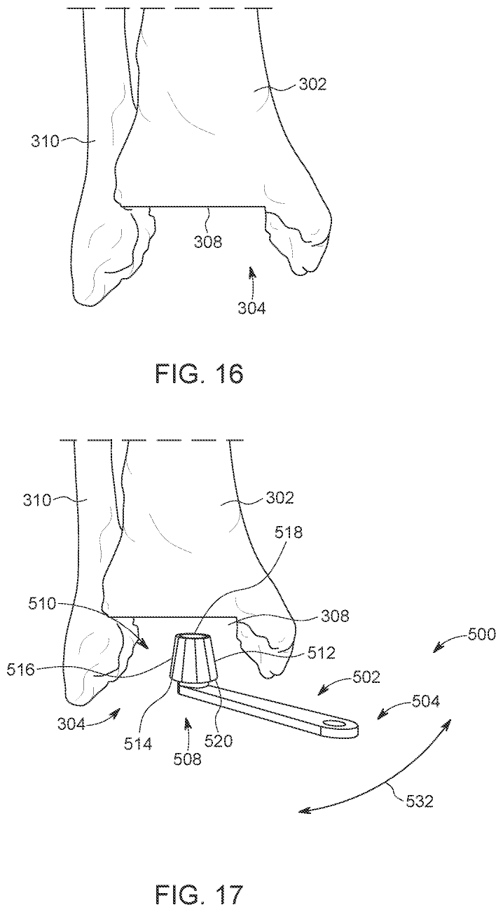

Referring now to FIGS. 16-20, the instrument 500 is shown relative to the exemplary tibia 302 and fibula 310 of a patient. The instrument 500 is shown to include a handle 502 having a first end 504 and a second end 508 which are disposed at opposite ends of the handle 502 as shown in the exemplary embodiment of FIG. 17. The handle 500 includes an engagement portion 510 arranged at (or adjacent to) the second end 508 of the handle 500, where the engagement portion 510 is coupled with the second end 508. In some aspects, the engagement portion 510 may be coupled with the second end 508 through a variety of means (e.g., pivotably coupled, rotatably coupled, etc.). Further, in some aspects the engagement portion 510 may be at least partially integral with the second end 508 of the handle 500.

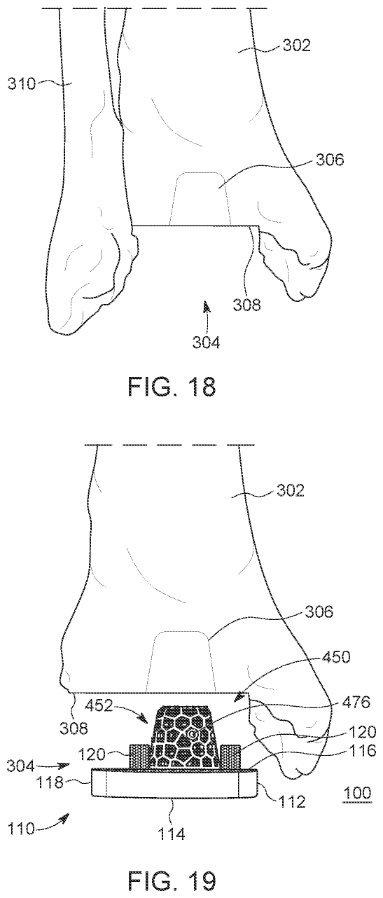

The engagement portion 510 is shown to include a body 512. The body 512 includes a bottom surface 514 and a top surface 518, as well as a lateral surface 516 extending between the bottom surface 514 and the top surface 518. The lateral surface 516 includes a plurality of engagement features 520 extending (e.g., projecting, protruding, etc.) from the lateral surface 516 at oblique and/or orthogonal angles relative to the lateral surface 516. Further, as shown the engagement features 520 are disposed substantially equidistant one another and extend vertically along the lateral surface 516 from the bottom surface 514 to the top surface 518 of the body 512. In some aspects, the engagement features 520 may extend vertically beyond the lateral surface 516 (e.g., past the bottom surface 514 and the top surface 518). Each of the engagement features 520 as shown includes a ridge protruding from the lateral surface 516 of the body 512, with said ridges each angled in a specific direction (e.g., similar to that shown with reference to the first engagement features 220 of the instrument 200 as shown in FIG. 6, the engagement features 520 are angled in a substantially counterclockwise direction relative to the engagement portion 510). The body 512 is configured to have a geometry similar to that of the body 452 of the augment 450 such that implementation of the instrument 500 (and the body 512) may ream or otherwise configure a bore 306 within a recess 308 (from resection to accommodate the tibial component 110) in the distal tibia 302 of the patient to accommodate at least a portion of the augment 450 (as shown with reference to FIGS. 18-20). Accordingly, the geometry of the body 512 of the engagement portion 510 has the general geometry of the frustum of a cone as defined previously herein with reference to the augment. It should be understood that the augment 150 may be configured such that it may be 3-D printed.

Referring to FIG. 17, the instrument 500 is shown just prior to engagement with the exemplary tibia 302 of a patient. The engagement portion 510 has been placed such that the top surface 518 will contact a distal surface of the recess 304 created in the distal tibia 302 (e.g., by resection). In the instance of a revision procedure, the engagement portion 510 may be placed in a portion of the distal tibia 302 which previously accommodated the stem 122 of the tibial component 110. In such an instance, the geometry of the engagement portion 510 allows for the engagement portion 510 to self-center when reaming a volume to accommodate the augment 450. That is to say that the geometry of the engagement portion 510 of the instrument 500 is configured to ream a volume centered about (and in this example, extending concentrically therefrom) the recess in the distal tibia that previously received the stem 122 (or other protrusions of other tibial components). In procedures that do not offer such a recess from a previous tibial component, the engagement portion is configured to center about a point identified by the physician and ultimately ream a volume with a geometry complimentary to that of the augment 450 such that said volume will receive the augment 450 (or at least a portion thereof) when implanted after coupling of the augment 450 with the tibial component 110. The instrument 500 as shown is a manual use instrument where a physician may manipulate the instrument 500 by the handle 502 about a semi-circular path 532 in a reciprocating nature to ream the desired volume in the distal tibia 302. In some aspects, the instrument 500 and/or portions thereof may be modified so as to couple with various other instruments (e.g., power tools/reamers). The instrument 500 may also be configured/designed such that it may be 3-D printed, thus eliminating the machining process common to many instruments.

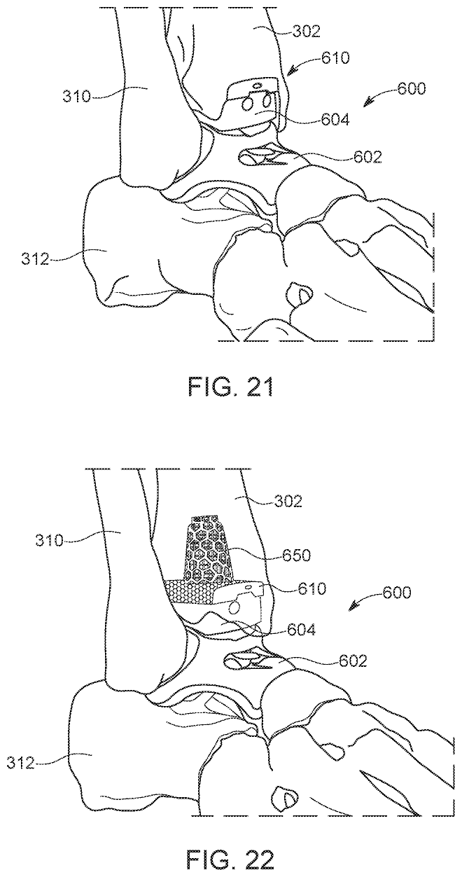

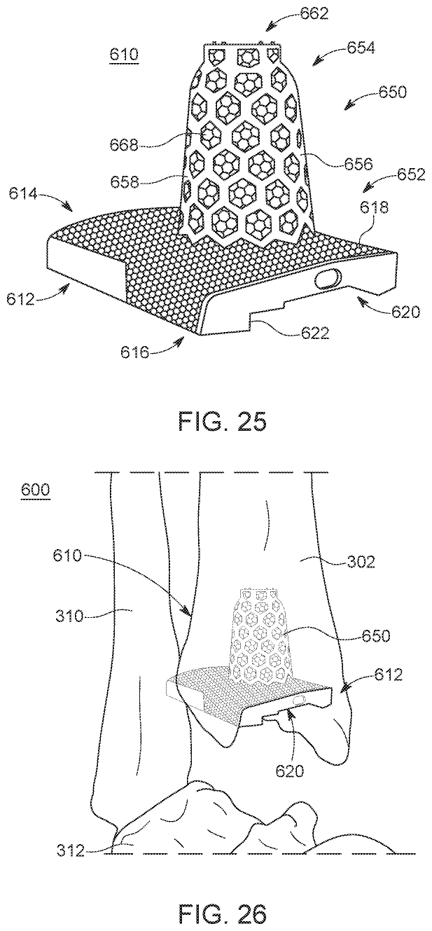

Referring now to FIGS. 21-26, an implant system 600 is shown, according to an exemplary embodiment of the present disclosure. The implant system 600 may include at least one of a talar component 602, an intermediate component 604, and/or a tibial component 610. In some aspects, the talar component 602 may be a talar spacer or other partial or total talar replacement implant, which may include one or more surfaces configured to interface with other bones/joints of the foot (e.g., articulate with and/or facilitate fusion with) and/or interface with (e.g., articulate with) other implant components such as the intermediate component 604. In some aspects, the talar component 602 may be configured to facilitate fusion with a calcaneus 312 of a patient, and may also be configured to interface with components of other implant systems (e.g., arthroplasty systems). Further, in some aspects the tibial component 610 may be the same as and/or similar to a tibial component of an ankle arthroplasty system configured to interface with the intermediate component 604 and thus provide compatibility with various talar components including the talar component 602 and/or talar components configured to couple with at least a portion of a native talus of a patient. It should be understood that the implant system 600 may be implemented as shown in FIGS. 21-26, or may be implemented in alternate configurations including various other components with cross-system compatibility, including but not limited to, those shown and described previously herein.

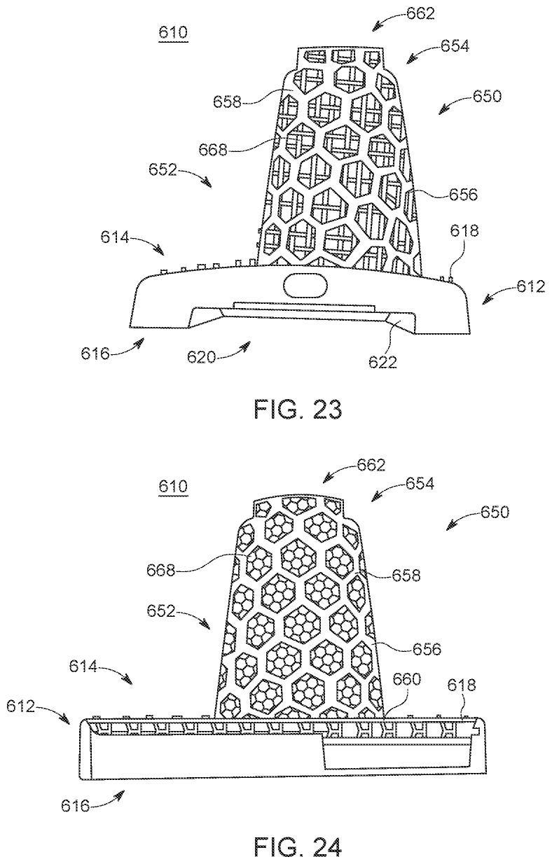

The tibial component 610 is shown to include a base portion 612 as well as bottom and top surfaces 614 and 616, respectively, where the top surface 614 is arranged opposite the base portion 612 from the bottom surface 616. The tibial component 610 is shown to include a texture 618 on the outer surface of the base portion 612 (including at least the top surface 614). The texture 118 may be configured to facilitate bone ingrowth and/or otherwise retain the tibial component 610 in the distal tibia. It should be noted that the tibial component 610 may be a standard tibial component (e.g., has no custom components, is sold off the shelf, etc.). The tibial component 610 is further shown to include a recess 620 disposed on the bottom surface 616 of the base portion 612 and, when shown in the position of FIG. 23, extends upward into the base portion 612 from the bottom surface 616. The recess 620 may include one or more engagement features 622 disposed on at least one surface therein, wherein said one or more engagement features 622 are configured to facilitate coupling (e.g., releasable, slidable, etc.) with another implant component. As shown, the engagement features 622 are configured to releasably couple with the intermediate component 604 where the engagement features 622 are configured as protrusions/grooves extending along at least a portion of the length of the recess 620 and configured to engage with complimentary protrusions/grooves of the intermediate component 604. In some aspects, the one or more engagement feature 622 may include alternate configurations and/or positioning within the recess 622. For example, the one or more engagement feature 622 may include a dovetail configuration or other various interface/engagement configurations. It should be understood that the tibial component 610 may be releasably couplable with various intermediate components, including but not limited to, the intermediate component 604, which is to say that the one or more engagement features 622 (and/or other features of the tibial component 604) may be configured to interface with various other implant components and/or implant systems.

An augment 650 is shown to be coupled with the top surface 616 of the tibial component 610. As shown, the augment is integral with the base portion 612, although in some aspects the augment may include one or more features (e.g., a central opening, etc.) configured to facilitate coupling with the base portion 612. As shown, the tibial component 610 includes a single augment 650, but in some embodiments multiple augments 650 of various geometries and/or sizes may be coupled with the tibial component 610. The augment 650 may be configured so as to provide a volume configured to occupy at least a portion of a complimentary volume of an irregularity in the distal tibia of the patient. As shown in FIGS. 23-26, the augment 650 is shown to be positioned off-center on the top surface 614 of the tibial component 610. In some aspects, the augment 650 may be selectively positioned about the tibial component 610 so as to address specific anatomy of a patient (e.g., a void in the distal tibia, a preference of a physician to bias the base portion 612 more anterior, posterior, medial, or lateral from a traditional, centered configuration, etc.). Further, in some aspects the augment 650 may be positioned so as to be received by an intramedullary canal of a tibia 302 of a patient, with the base portion 612 of the tibial component 610 positioned based on the position of the augment 650 relative to the base portion 612. Further, in some aspects the augment 650 may be configured to occupy the entirety of the volume of an irregularity or may be sized so as to have a volume greater than the irregularity (e.g., to facilitate a desired fit). Furthermore, and as shown and described subsequently herein, the augment 650 may include one or more complimentary instruments configured to facilitate implantation of the tibial component 610 with the augment 650 (e.g., a reamer to ream an irregularity into a volume that will receive the augment 650, etc.). In some aspects, the augment may be of a custom size based on an irregularity identified and measured via imaging data. Further, in some aspects, the augment 650 may be selected by a physician from a library of augments 650 (and/or augment trials) provided in a surgical kit, where each augment 650 has a different size/shape/geometry so as to address various irregularities in the distal tibia of the patient. It should be noted that an appropriate version of the augment 650 for an irregularity (or potentially multiple irregularities) may require manipulation of the geometry of the irregularity in order for the augment 650 to fit as desired.

As shown in FIGS. 23-26, the augment 650 has a bottom portion 652 and a top portion 654. The geometry of the bottom portion 652 of the augment 650 is that of a frustum of a cone shape (e.g., where a frustum is a portion of a solid that lies between two parallel lines cutting said solid), and the geometry of the top portion 654 is that of a cylinder with a diameter greater than the height. The cylindrical geometry of the top portion 654, as described subsequently herein, corresponds to a complimentary cylindrical volume formed in the distal portion of the tibia prior to implantation. Such cylindrical geometry (both of the top portion 654 and said corresponding volume) is configured to facilitate coupling of the augment 650 (and other components of the system as shown and described) with the distal tibia and retention thereof. It should be noted that in some aspects the geometry of the augment 650 may be variable (e.g., the bottom portion 652 may have a lesser height/volume/surface area than that of the top portion 654, the bottom portion 652 and top portion 654 may each have alternate geometries, etc.).

The augment 650 further includes an exterior surface 656, a bottom surface 660, and a top surface 662. The exterior surface 656 extends between the bottom surface 660 and the top surface 662. As shown, the bottom surface 660 is integral with the top surface 616 of the tibial component 610. The top surface 662 is arranged opposite the augment 650 from the bottom surface and is an upper surface of the top portion 654 of the augment 650. In some aspects, the augment 650 may include an interior surface defining a volume of a central opening of the augment 650, although in other aspects the central portion of the augment 650 may be solid. The augment 650 also has a pair of porous structures (e.g., lattice structures, web structures, nodal structures, mesh structures, etc.) including a first porous structure 658 and a second porous structure 668. As shown, the first porous structure 658 is arranged proud relative to the second porous structure 668, where the first porous structure 658 defines an outer surface of the augment 650 for all of the aforementioned surfaces. The first porous structure 658 is shown to have a greater porosity (e.g., more porous) than that of the second porous structure 668. As shown, the first porous structure is a substantially randomized structure (while still satisfying various design inputs and structural parameters) whereas the second porous structure has a grid-shaped geometry (e.g., with substantially orthogonal components). It should be understood that the first and second porous structures 658, 668 as shown and described herein, are exemplary and may be modified for various embodiments of the augment 650. For example, a particular irregularity in the distal tibia of a patient may be conducive to one or more porous structures with greater or lesser porosities than those shown in the exemplary augment 650. Ultimately, the augment 650 may include an alternate number of porous structures or alternate porous structures, or an alternate arrangement of two or more porous structures.

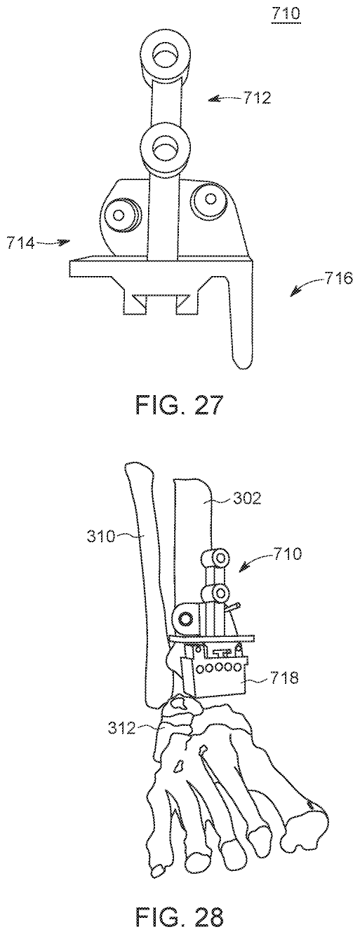

Referring now to FIGS. 27-28, an instrument 710 is shown, according to an exemplary embodiment of the present disclosure. The instrument 710 as shown may be a guide or other instrument configured to facilitate and/or guide drill holes and/or cuts to the distal tibia 304 of a patient in order to prepare the distal tibia 304 of a patient for implantation of at least a portion of the system 600 (e.g., the tibial component 610). In some aspects, the instrument 710 may be color-coded in order to indicate a specific step in a procedure, or maybe one of multiple instruments 710 of various sizes included in a surgical kit where color coding indicates size of the instrument 710. The instrument 710 may also be a patient specific instrument/guide generated (e.g., 3-D printed, etc.) specifically to fit an anatomy of a patient based on imaging data and/or modeling. The instrument 710 as shown includes a vertical protrusion 712 including at least one opening (as shown, two openings) configured to guide a drill or other instrument and, in some aspects, placement of pins or guidewires. The instrument 710 further includes at least one opening in a central portion 714 thereof similarly configured to guide a drill for the placement of pins and/or wires. A protrusion 716 is shown to extend from the central portion 714 in a direction substantially opposite that the protrusion 712, where the protrusion 716 may be configured to facilitate manipulation of the instrument 710 and/or guide various other actions by a physician. In some aspects, the instrument 710 may be releasably couplable with a portion of the anatomy of a patient (e.g., via pins, slidably coupled, etc.).

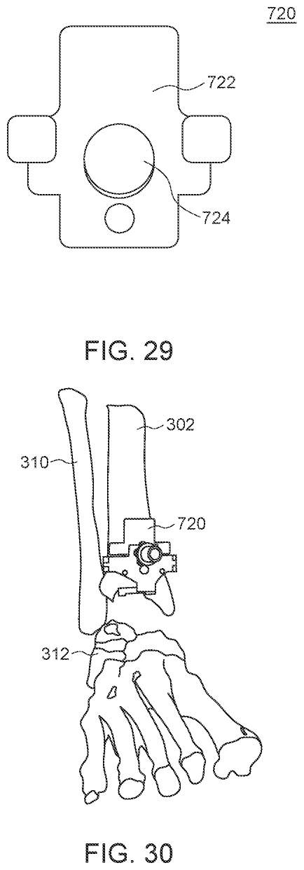

Referring now to FIGS. 29-30, an instrument 720 is shown, according to an exemplary embodiment of the present disclosure. Similar to the instrument 710, the instrument 720 may be a guide or other instrument configured to facilitate and/or guide drill holes and/or cuts to the distal tibia 304 of a patient in order to prepare the distal tibia 304 of a patient for implantation of at least a portion of the system 600 (e.g., the tibial component 610). Further, the instrument 720 may be configured to releasably couple with at least a portion of the anatomy of the patient. In some aspects, the instrument 720 may be color-coded in order to indicate a specific step in a procedure, or maybe one of multiple instruments 720 of various sizes included in a surgical kit where color coding indicates size of the instrument 720. The instrument 720 may also be a patient specific instrument/guide generated (e.g., 3-D printed, etc.) specifically to fit an anatomy of a patient based on imaging data and/or modeling. The instrument 720 includes a base portion 722 which includes an opening 724 positioned substantially centrally relative to the base portion 722. In some aspects, the opening 724 may be positioned relative to a portion of the anatomy of the patient to which a specific operation may be performed. For example, the instrument 720 may be generated with the opening 724 in a specific position such that, when releasably coupled with a patient, the opening 724 aligns with a portion of the anatomy (e.g., an intramedullary canal of the distal tibia) and a physician may insert at least a portion of a reamer within the opening 724 so as to ensure reaming of the desired portion of the anatomy. In some aspects, the instrument 720 may be releasably couplable with a portion of the anatomy of a patient (e.g., via pins, slidably coupled, etc.).

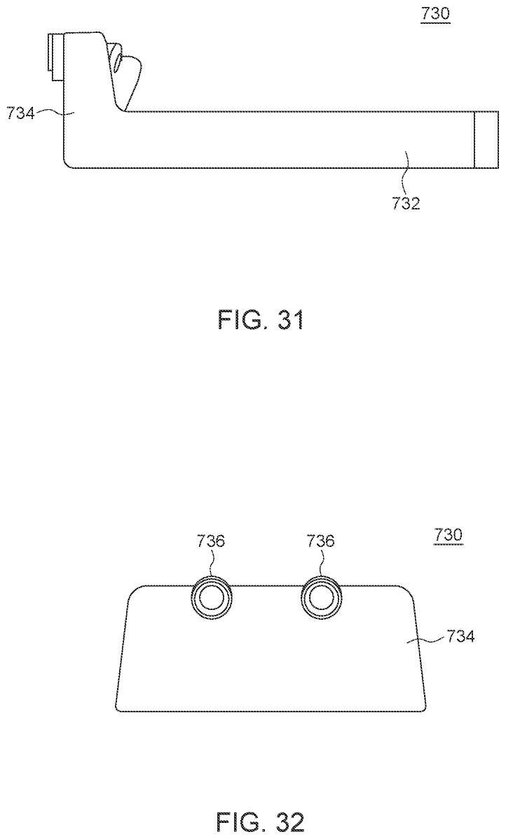

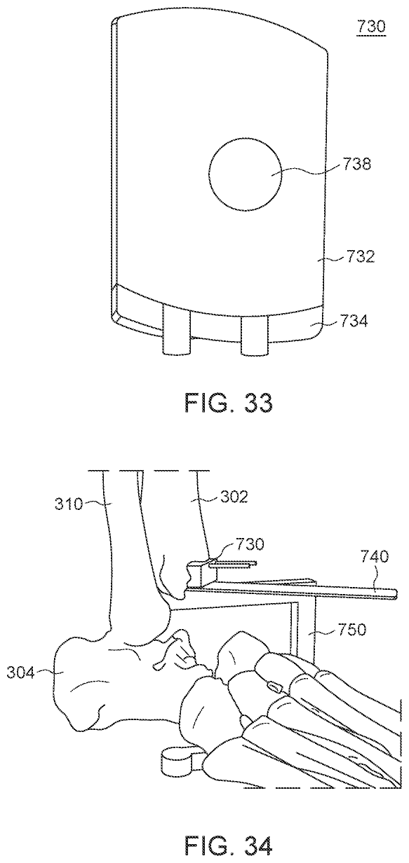

Referring now to FIGS. 31-34, an instrument 730 is shown, according to an exemplary embodiment of the present disclosure. Similar to the instruments 710, 720, the instrument 730 may be a guide or other instrument configured to facilitate and/or guide drill holes and/or cuts to the distal tibia 304 of a patient in order to prepare the distal tibia 304 of a patient for implantation of at least a portion of the system 600 (e.g., the tibial component 610). Further, the instrument 730 may be configured to releasably couple with at least a portion of the anatomy of the patient. In some aspects, the instrument 730 may be color-coded in order to indicate a specific step in a procedure, or maybe one of multiple instruments 730 of various sizes included in a surgical kit where color coding indicates size of the instrument 730. The instrument 730 may also be a patient specific instrument/guide generated (e.g., 3-D printed, etc.) specifically to fit an anatomy of a patient based on imaging data and/or modeling. The instrument 730 includes a base portion 732 which includes an opening 738 positioned substantially centrally relative to the base portion 732 (for example, in a central portion of the base portion 732 which may or may not be “centered” relative to the instrument 730). In some aspects, the opening 738 may be positioned on the instrument 730 relative to a portion of the anatomy of the patient to which a specific operation may be performed. For example, the instrument 730 may be generated with the opening 738 in a specific position such that, when releasably coupled with a patient, the opening 738 aligns with a portion of the anatomy (e.g., an intramedullary canal of the distal tibia) and a physician may insert at least a portion of a reamer within the opening 738 so as to ensure reaming of the desired portion of the anatomy. The instrument 730 is also shown to include a protrusion 734 extending from the base portion 732 in a substantially orthogonal direction and from an end portion of the base portion 732. The protrusion 734 is shown to include at least one opening 736 (as shown, two openings) configured to guide drill holes and/or placement of wires/pins by a physician.

As shown in FIG. 34, the instrument 730 may be positioned on the distal tibia 304 of a patient such that the at least one opening 736 is disposed on an anterior portion thereof. Further, the instrument 730 may also be implemented in conjunction with other instruments, for example the instrument 750 which may be configured to retain the instrument 730 and/or one or more portions of the anatomy of a patient in a desired location during a procedure. In some aspects, the instrument 730 may be releasably couplable with a portion of the anatomy of a patient (e.g., via pins, slidably coupled, etc.).

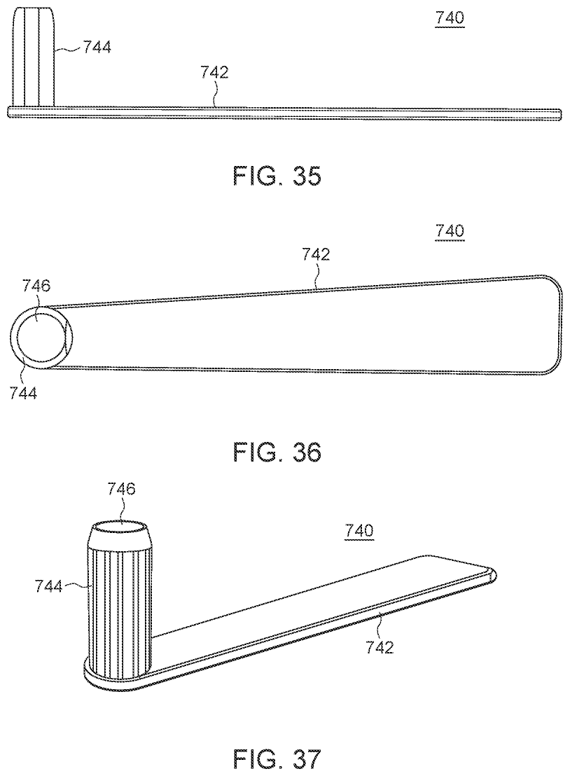

Referring now to FIGS. 34-38, an instrument 740 is shown, according to an exemplary embodiment of the present disclosure. The instrument 740 is shown relative to the distal tibia 302 and fibula 310 of a patient. The instrument 740 is shown to include a handle 742 and a head 744 positioned at an end of the handle 742. As shown, the handle 742 has a substantially tapered configuration with a wider lateral dimension at the end thereof opposite the head 744. The instrument 740 and portions thereof may be the same as and/or similar to the instrument 500 shown and described herein previously and, as such, may also be implemented in the same and/or similar fashion to that shown in at least FIG. 17 herein and will not be repeated for brevity sake. The instruments 720, 730 may be configured to have one or more geometries that accommodate one or more features of the instrument 740 and/or other similar instruments (e.g., as shown in FIG. 38). For example, the openings 724, 738 may be configured to receive at least a portion of the head 744 of the instrument 740 therein so as to guide reaming of the distal tibia 304 by a physician. In some aspects, the instrument 740 may be configured to a specific anatomy of a patient (e.g., the head 744 may be taller, have a greater lateral dimension, etc.) to accommodate said anatomy.

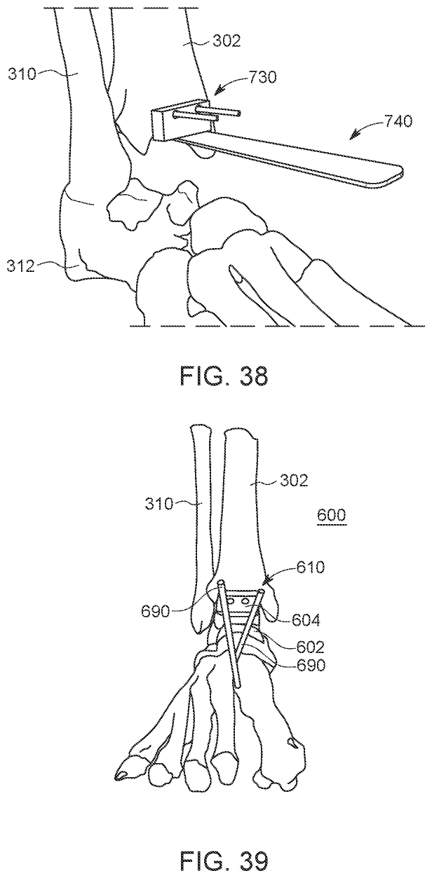

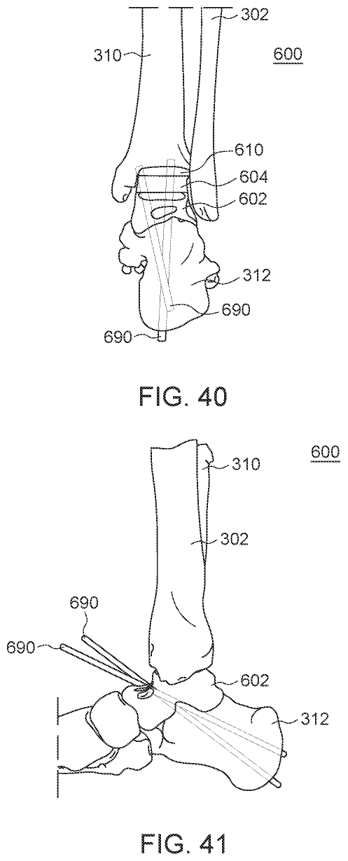

Referring now to FIGS. 39-41, the implant system 600 is shown in an implanted state adjacent the tibia, fibula, and calcaneus 304, 310, 312 of a patient, according to an exemplary embodiment of the present disclosure. The tibial component 610 is shown coupled with the distal tibia 304 of a patient and, while not shown, the augment 650 is disposed at least partially within the tibia 304 (e.g., in the intramedullary canal). The intermediate component 604 is shown to be releasably coupled with the tibial component 610 (via the one or more engagement features 622, although not shown here) such that the intermediate component 604 is retained in a position substantially inferior/plantar relative to the tibial component 610. The talar component 602 is shown to be positioned inferior/plantar relative to the intermediate component 604 such that an upper surface of the talar component 602 may interface with a lower surface of the intermediate component. Further, a pair of trajectories 690 are shown extending through at least a portion of the talar component 602 and through the calcaneus 312. The pair of trajectories 690 corresponds to trajectories of fixation elements (e.g., fasteners, screws, compression screws, lag screws, etc.) configured to facilitate placement of the talar component 604 with the calcaneus 312 (e.g., arthrodesis, arthroeresis, etc.). In some aspects, the subtalar joint between the talar component 602 and the calcaneus 312 may be fused in performing a procedure that includes implanting one or more components of the implant system 600, although this may not be included in all such procedures.

It should be understood that the implant systems 100, 600 as well as any components thereof and/or instruments shown and described herein should be considered interchangeable and cross-compatible. That is to say that the instrument 730 may be implemented in conjunction with the instrument 500 and the implant system 100, or the implant system 600 may be implemented in conjunction with the instrument 500 and the instrument 710. Further, it should be understood that any of the components shown and described herein may be configured specific to an anatomy of a patient. For example, the augment 650 may have height and lateral dimension corresponding to dimensions of a tibial void or the intramedullary canal of a patient, or the head 744 of the instrument 700 may be sized according to similar anatomical dimensions of a patient.

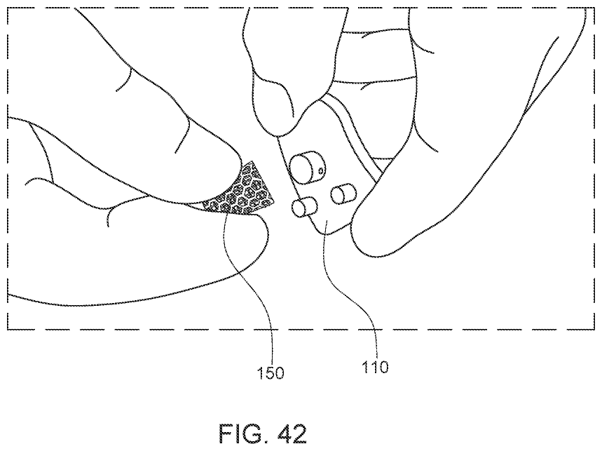

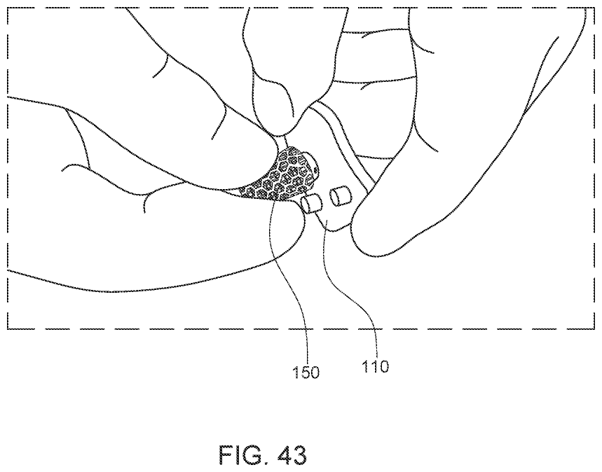

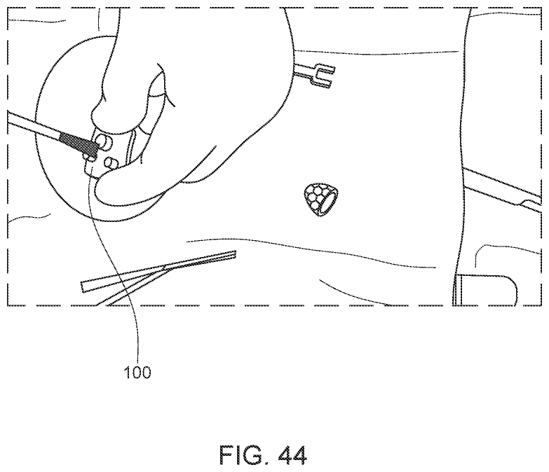

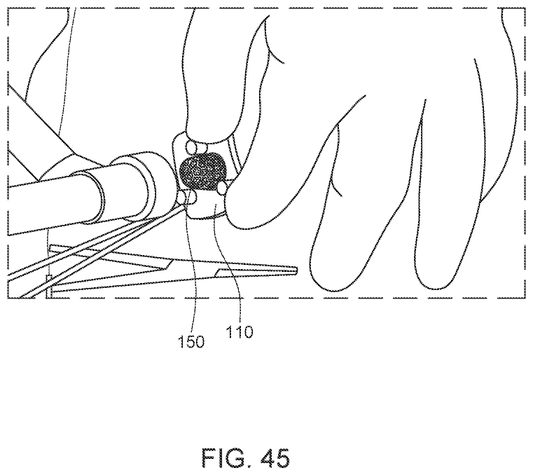

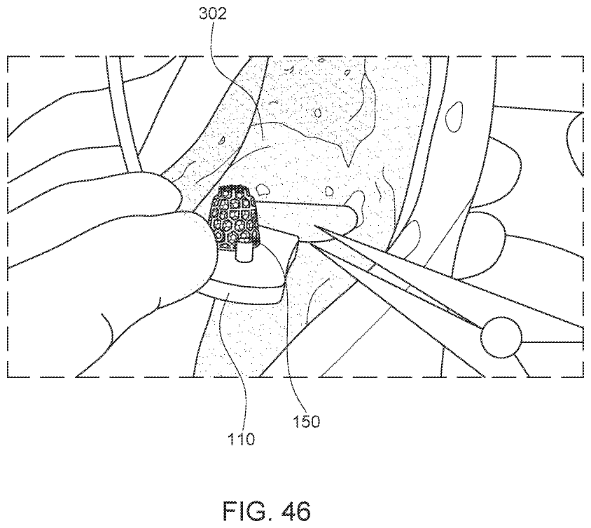

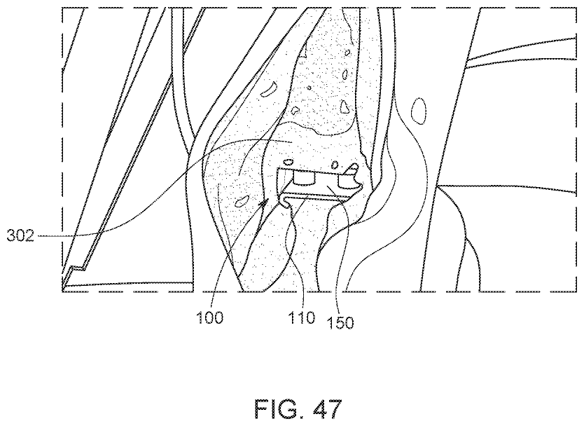

FIGS. 42-47 show the implant system 100 being assembled by a physician prior to implantation. FIG. 42, shows a physician preparing to couple the augment 150 to the tibial component. FIG. 43 shows the physician connecting the augment 150 to the tibial component 110. FIG. 44 shows the assembled implant system 100 being prepared for implantation into the distal tibia 302 of the patient. FIG. 45 shows the physician using a tool to secure the augment 150 to the tibial component 110. FIG. 46 shows the implant system 100 adjacent to the distal tibia 302 prior to implantation. FIG. 47 shows the implant system 100 implanted into the distal tibia 302.

It is understood by one skilled in the art that the implant system 100, although shown as a two piece construct could be one piece manufactured out of titanium or another biocompatible metal material. The implant system 100, either as a one piece construct or a two piece construct (i.e., tibial component and augment) may be manufactured using a 3-D printer or some additive manufacturing process or machined out of bulk metal bars. Although the implant system 100 as described herein is directed towards an ankle replacement implant, it is contemplated that the augment element could be used with other joint implants to stabilize the implant post-operatively. For example, the augment 150 could be using in a total shoulder arthroplasty system, an acetabulum cup, either the femoral or tibial component of a total knee and virtually all other prosthetic joint devices that are inserted within a bone of a patient.

Although the implant system as explained in relation to its preferred embodiment, it is to be understood that many other possible modifications and variations can be made without departing from the spirit and scope of the invention.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprise” (and any form of comprise, such as “comprises” and “comprising”), “have” (and any form of have, such as “has”, and “having”), “include” (and any form of include, such as “includes” and “including”), and “contain” (and any form of contain, such as “contains” and “containing”) are open-ended linking verbs. As a result, a method or device that “comprises,” “has,” “includes,” or “contains” one or more steps or elements possesses those one or more steps or elements, but is not limited to possessing only those one or more steps or elements. Likewise, a step of a method or an element of a device that “comprises,” “has,” “includes,” or “contains” one or more features possesses those one or more features, but is not limited to possessing only those one or more features. Furthermore, a device or structure that is configured in a certain way is configured in at least that way, but may also be configured in ways that are not listed.

The invention has been described with reference to the preferred embodiments. It will be understood that the architectural and operational embodiments described herein are exemplary of a plurality of possible arrangements to provide the same general features, characteristics, and general system operation. Modifications and alterations will occur to others upon a reading and understanding of the preceding detailed description. It is intended that the invention be construed as including all such modifications and alterations.

What is claimed is:

1. A system for augmentation of a tibial component, the system comprising:
an augmentation component, comprising:
a first porous structure;
a second porous structure;
a top portion having a top surface;
a bottom portion having a bottom surface arranged opposite the augmentation component from the top surface;
a tibial component; and
an instrument, comprising:
an engagement portion having a geometry complimentary to that of the augmentation component.

2. The system of claim 1, wherein the augmentation component is couplable with a surface of the tibial component.

3. The system of claim 1, wherein the first porous structure is positioned proud relative to the second porous structure.

4. The system of claim 3, wherein the first porous structure comprises a first porosity and the second porous structure comprises a second porosity.

5. The system of claim 4, wherein the first porosity is greater than the second porosity.

6. The system of claim 1, wherein the top portion of the augmentation component is positioned superior relative to the bottom portion of the augmentation component.

7. The system of claim 6, wherein the bottom portion comprises at least one first lateral dimension and the top portion comprises at least one second lateral dimension.

8. The system of claim 7, wherein the at least one first lateral dimension is a plurality of lateral dimensions, wherein each of the plurality of lateral dimensions are greater than the at least one second lateral dimension.

9. The system of claim 7, wherein the at least one first lateral dimension comprises a plurality of lateral dimensions, wherein the plurality of lateral dimensions decrease in magnitude from the bottom surface of the bottom portion toward a top surface of the bottom portion.

10. The system of claim 1, wherein the augmentation component comprises an interior volume configured to receive a protrusion disposed on a surface of the tibial component.

11. The system of claim 1, wherein the instrument is a reaming instrument configured to remove a volume of bone that corresponds to a volume of the augmentation component.

12. The system of claim 11, wherein the instrument further comprises:
a handle;
an engagement component coupled with the handle, comprising:
a lower engagement portion, comprising:
a plurality of first engagement features disposed on at least a portion of at least a lateral surface and a top surface of the lower engagement portion; and
an upper engagement portion, wherein the upper engagement portion is positioned superior to the lower engagement portion and comprises:
a plurality of second engagement features disposed on at least a portion of at least a lateral surface and a top surface of the upper engagement portion.

13. The system of claim 12, wherein the plurality of first engagement features and the plurality of second engagement features protrude from the surfaces of the lower engagement portion and the upper engagement portion.

14. An augmentation component for a tibial component, the augmentation component comprising:
a top portion comprising at least one lateral dimension;
a bottom portion comprising a plurality of lateral dimensions that decrease in magnitude from a bottom surface of the bottom portion to a top surface of the bottom portion.

15. The augmentation component of claim 14, wherein the top portion is positioned superior to and is integral with the bottom portion.

16. The augmentation component of claim 15, further comprising:
a central volume extending from the bottom surface of the bottom portion into the augmentation component, the central volume comprising an interior surface.

17. The augmentation component of claim 16, further comprising:
a bore extending from an exterior surface of the augmentation component through to the interior surface of the augmentation component and providing fluid communication from the central volume to the exterior surface of the augmentation component.

18. The augmentation component of claim 14, further comprising:
a first porous structure comprising a first porosity; and
a second porous structure comprising a second porosity, wherein the first porous structure is positioned proud relative to the second porous structure.

19. The augmentation component of claim 18, wherein the first porosity is greater than the second porosity.

20. A method of augmenting a tibial component, comprising:
collecting imaging data from a patient;
identifying, from the imaging data, a void in a distal portion of a tibia of the patient;
obtaining an augmentation component comprising a volume that corresponds to a volume of the void of the patient;
coupling the augmentation component with a tibial component; and
implanting the tibial component such that the augmentation component occupies at least a portion of the void of the patient.

21. An implant kit, comprising:
a tibial component;
an instrument having a cutting head; and
a plurality of different configured augmentation components for coupling to the tibial component, wherein the cutting head is configured to prepare a bone to receive at least one of the plurality of augmentation components.
